(19) United States
(12) Patent Application Publication
(10) Pub. No.: US 2009/0149945 A1
(43) Pub. Date: Jun. 11, 2009

(54) PROSTHETIC SUPPORT FOR FLACCID ARTERIAL SEGMENTS

(75) Inventor: Kelly Pike, Half Moon Bay, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: Abbott Carciovascular Systems Inc., a California corporation, Santa Clara, CA (US)

(21) Appl. No.: 11/951,821

(22) Filed: Dec. 6, 2007

(51) Int. Cl. A61F 2/06

(52) U.S. Cl. 623/1.36

(57) ABSTRACT

A supplemental elasticity device is attached to an artery in the knee to replace lost elastic behavior. The supplemental elasticity device is attached near both ends of the vessel section which it is intended to compress. In this case, the vessel section extends from the adductor canal to below the knee joint. Supplemental elasticity device fixation can be achieved by tabs, spikes or hooks extending from the supplemental elasticity device or increased friction between the supplemental elasticity device and vessel wall, or a combination of both. To assist with maintaining hemostasis, the fixation points may include fabric patches on the supplemental elasticity device surface. The supplemental elasticity device can be deployed in an un-stretched or nominal length when the leg is bent. It is also possible to deploy when the leg is straight if the supplemental elasticity device is in an elongated configuration during deployment. In one embodiment, the supplemental elasticity device has the ability to elongate 15% of its length and return to its nominal length for the life of the supplemental elasticity device. For an annual duty cycle of 62,000 cycles per year, a ten year life would require the supplemental elasticity device to remain intact for 620,000 cycles.

### BACKGROUND OF THE INVENTION

Like many other elastin-containing anatomical structures, arteries lose their spring-like character with advancing age. Generally, this leads to a reduction of arterial radial compliance. For the superficial femoral leg artery (SFA) and popliteal leg arteries, which stretch and recover during locomotion, this can lead to arterial kinking.

The SFA and popliteal leg arteries are stretched out when the leg is straight such as when a person is standing. The vessels return to their nominal length when the knee is bent to 70-90 degrees, as occurs when a person is sitting. A similar stretch and recover cycle occurs to the leg arteries during walking or stair climbing. But if a vessel suffers a loss of its elastic behavior, the stretched vessel will not recover and elongation can become permanent.

It has been estimated that the SFA-popliteal arterial segment may stretch as much as 15% during a knee bending cycle. The treated arterial segment can be as long as 200 mm. This requires the artery to stretch and recover as much as 30 mm of length. Unable to contract to the original length, the arterial slack must accommodate the forces shortening it along its length. The result is bunching and kinking of the artery. The location of the artery bunching and kinking is across the space behind the knee joint where there is no supporting muscle capsule surrounding the artery.

FIG. 1 illustrates a leg artery 2 in a relatively unstretched configuration when the knee is bent. FIG. 2 illustrates the same leg artery 2 in a stretched out configuration when the leg is straightened. After the stretching of FIG. 2, an older artery 2′ that has lost elasticity will kink and bunch as in FIG. 3.

What has been needed and heretofore unavailable is a method of providing supplemental elasticity to vessels in body joints, to reduce bunching and/or kinking. The present invention satisfies this need.

### SUMMARY OF THE INVENTION

The present invention relates to methods and devices for providing supplemental elasticity to a popliteal leg artery and/or a superficial femoral leg artery. One aspect of the invention relates to a method for prosthetic support of flaccid arterial segments. A supplemental elasticity device is implanted into one of a popliteal leg artery and a superficial femoral leg artery. The supplemental elasticity device is then attached into place within the artery.

In one embodiment, the supplemental elasticity device has various adjacent cylindrical elements, each having a circumference extending around a longitudinal supplemental elasticity device axis. Each element is independently expandable in the radial direction. The elements are arranged in alignment along the longitudinal supplemental elasticity device axis. At least one interconnecting member extends between adjacent cylindrical elements and connects them to one another. There are also protrusions on one or both ends of the supplemental elasticity device for attaching the supplemental elasticity device to the body lumen.

The protrusions may optionally be formed of a unitary structure, and the step of attaching the supplemental elasticity device into place includes radially expanding the supplemental elasticity device to extend the protrusions outwardly to engage the wall of the body lumen.

The method may include various other features. For example, the supplemental elasticity device may include hooks. The step of attaching the supplemental elasticity device into place then includes engaging a wall of the body lumen with at least one hook. The supplemental elasticity device may optionally include tabs, instead of or in addition to hooks, and the step of attaching the supplemental elasticity device into place includes engaging a wall of the body lumen with at least one tab. The protrusions may include barbs, and the step of attaching the supplemental elasticity device into place includes engaging a wall of the body lumen with at least one barb.

The supplemental elasticity device may be made of any of a variety of materials, such as superelastic NITINOL or another superelastic material, stainless steel, a biocompatible polymer, and/or other materials, including cobalt-chromium, titanium, nickel-titanium, tantalum, gold, platinum, nickel-titanium-platinum, and other similar metal alloys known in the art, or from a polymeric material known in the art for making stents.

One embodiment of a prosthetic support for supporting flaccid arterial segments includes a plurality of adjacent cylindrical elements each having a circumference extending around a longitudinal supplemental elasticity device axis. Each element is substantially independently expandable in the radial direction, and the elements are arranged in alignment along the longitudinal supplemental elasticity device axis. The cylindrical elements are formed in a generally serpentine wave pattern transverse to the longitudinal axis and contain alternating peaks and valleys. At least one interconnecting member extends between adjacent cylindrical elements and connecting them to one another. A plurality of protrusions on the supplemental elasticity device serve to attach the supplemental elasticity device to the body lumen.

In one embodiment, the supplemental elasticity device is capable of enduring elongation cycles in which the supplemental elasticity device is elongated up to 15% of its normal length when a patient straightens a leg, and the supplemental elasticity device is then returned to a normal length when the patient bends the knee, throughout a life of at least 620,000 elongation cycles.

Another embodiment of the invention relates to a method of manufacture, in which a prosthetic support is cut from a tube of material. Protrusions on at least one end of the prosthetic support are also cut. The protrusions are then bent to extend outwardly from the prosthetic support.

The protrusions may be bent into any of a variety of configurations, either alone or in combination. For example, the protrusions may be bent into hooks, barbs, tabs, or any other configuration suitable for securing the supplemental elasticity device within a lumen. In one particular embodiment, the protrusions are cut as members each having a frame that defines an interior, and a tab member extends from the frame toward the interior. The tab member may be bent to extend outwardly from the frame.

Other features and advantages of the present invention will become more apparent from the following detailed description of the invention, when taken in conjunction with the accompanying exemplary drawings.

### BRIEF DESCRIPTION OF THE DRAWINGS

FIG. 1 illustrates in a general way the popliteal artery as it appears when the knee is bent.

FIG. 2 illustrates in a general way the popliteal artery of FIG. 1, in a stretched and straighten configuration when the knee is straight.

FIG. 3 illustrates popliteal arterial kinking with the knee bent.

FIG. 4 illustrates the popliteal artery with the knee straight and a supplemental elasticity supplemental elasticity device placed therein.

FIG. 5 illustrates the popliteal artery of FIG. 4 with the knee bent and the supplemental elasticity supplemental elasticity device preventing arterial kinking.

FIG. 6 illustrates one embodiment of a hook.

FIG. 7 illustrates one embodiment of a tab.

FIG. 8 is a perspective view depicting the pattern of a portion of a supplemental elasticity device.

FIG. 9 is a plan view depicting a supplemental elasticity device having barbs at one end.

FIG. 10 is a perspective view of a portion of an expanded configuration of the supplemental elasticity device of FIG. 9.

FIG. 11 is a perspective view of a portion of a supplemental elasticity device.

FIG. 12A is a compressed plan view depicting the pattern of a supplemental elasticity device including tabs for attachment means.

FIG. 12B is a view depicting the pattern of a supplemental elasticity device including hooks for attachment means.

FIG. 12C is a detail view of a tab of FIG. 12A.

FIG. 12D is a detail view of a hook of FIG. 12B.

FIG. 13 is a plan view of a supplemental elasticity device depicting alternative attachment means.

FIG. 14 is a detail view of a supplemental elasticity device with hooks with barbs of FIG. 13.

FIG. 15 is an elevational view, partially in section, of a supplemental elasticity device embodying features of the invention which is mounted on a delivery catheter and disposed within an artery.

FIG. 16 is an elevational view, partially in section, similar to that shown in FIG. 15, wherein the supplemental elasticity device is expanded within an artery.

FIG. 17 is an elevational view, partially in section, showing the expanded supplemental elasticity device within the artery after withdrawal of the delivery catheter.

### DETAILED DESCRIPTION OF THE PREFERRED EMBODIMENTS

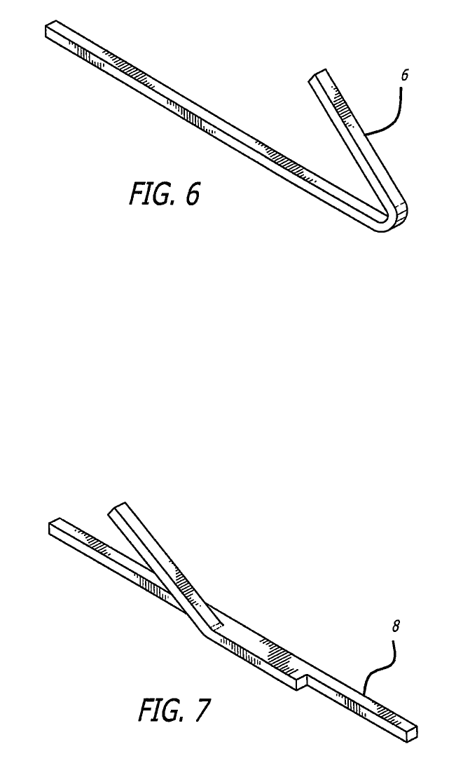

To overcome the kinking and bunching of FIG. 3, according to the present invention a stretchable supplemental elasticity device 4 may be implanted and fixed into place within the artery 2. FIG. 4 shows one nonlimiting example of such a supplemental elasticity device 4 with the ends of the supplemental elasticity device 2 affixed to the walls of the artery by way of hooks (e.g., for general illustration only, FIG. 6), tabs (e.g., FIG. 7) or other means, such as fabric or other friction-causing material. The supplemental elasticity device of FIG. 4 has been stretched somewhat, and acts as a spring. When the knee is bent and the artery returns to a normal length, the spring action of the supplemental elasticity device as the supplemental elasticity device returns to normal length serves to prevent kinking and bunching of the artery. The supplemental elasticity device may be any of a variety of different designs, so long as the desired spring effect is achieved, so as to provide supplemental elasticity to the artery. In an alternative embodiment, a type of spring that is not a supplemental elasticity device may be used.

This supplemental elasticity device should be attached near both ends of the vessel section which it is intended to compress. In this case, the vessel section extends from the adductor canal to below the knee joint. Supplemental elasticity device fixation can be achieved by tabs, barbs, spikes and/or hooks extending from the supplemental elasticity device, or from increased friction between the supplemental elasticity device and vessel wall, or a combination of both. To assist with maintaining hemostasis, the fixation points may optionally include fabric patches on the supplemental elasticity device surface.

The supplemental elasticity device can be deployed in an un-stretched or nominal length when the leg is bent. It is also possible to deploy when the leg is straight if the supplemental elasticity device is in an elongated configuration during deployment.

The supplemental elasticity device could resemble a self expanding supplemental elasticity device similar to Guidant's Dynalink 0.035 or Absolute 0.035 supplemental elasticity device delivery system products. In one embodiment, the supplemental elasticity device has the ability to elongate 15% of its length and return to its nominal length for the life of the supplemental elasticity device. For an annual duty cycle of 62,000 cycles per year, a ten year life would require the supplemental elasticity device to remain intact for 620,000 cycles. The contraction force of the supplemental elasticity device should approximate the healthy vessel properties. It is possible that the supplemental elasticity device contraction force can be less than a healthy vessel's when the supplemental elasticity device is only required to supplement a partially compromised vessel contraction. Alternatively it is possible for the supplemental elasticity device to have a higher contraction force and greater shortening range when the supplemental elasticity device length is less than the target vessel segment.

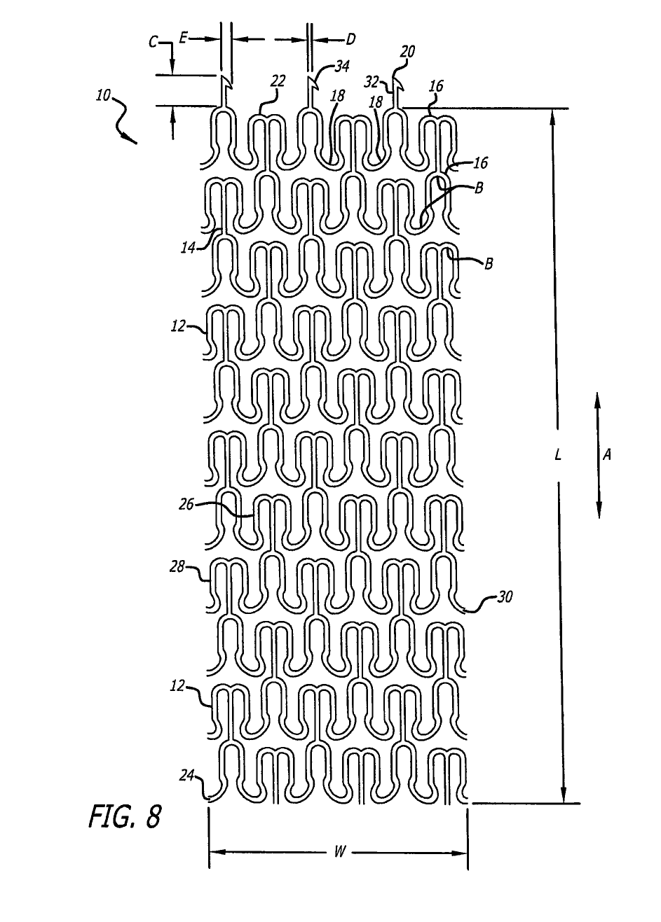

Considering now a non-limiting example of one embodiment of a supplemental elasticity device according to the present invention, FIG. 8 depicts the pattern of a supplemental elasticity device 10 incorporating features of the invention. The supplemental elasticity device generally is a spring. In one embodiment, the supplemental elasticity device is a stent, or is stent-like, comprised of a plurality of cylindrical rings 12 which are not so tightly spaced as to inhibit the flexibility of the combination.

The cylindrical rings are connected to each other by connecting members 14. Each cylindrical ring is characterized by serpentine or wave pattern, having a series of alternating peaks 16 and valleys 18. The degrees of curvature indicated by arrows B along adjacent peaks and valleys are different and, preferably, the pattern of each cylindrical ring is in phase with the pattern of every other cylindrical ring. Attachment elements 20, shown in FIG. 8 in the form of barbs, can be provided on first end 22 of the supplemental elasticity device, to engage with the arterial wall when the supplemental elasticity device is deployed. Second end 24 of the supplemental elasticity device may be attached to an additional supplemental elasticity device, to form a chain of such devices, and/or may also have hooks, barbs, or the like for securing the end to the interior of the lumen.

When two supplemental elasticity devices 10 are used in combination, only the supplemental elasticity device situated at the most distal end need be provided with attachment elements in order to adequately anchor the combination to the vessel. In FIG. 8, the configuration of the barbs is such that each is positioned at every other peak 18 that is at first end 22 of the supplemental elasticity device, which will comprise the most distal end of the supplemental elasticity device when it is fully formed and oriented for deployment. Each barb has shaft portion 32 extending outwardly from the distal most cylindrical ring, and a pointed portion 34 extending from the shaft.

The properties of superelastic NITINOL (Nitinol Devices and Components) make it a preferred material for the supplemental elasticity device. Other biocompatible materials such as stainless steel or a suitable polymeric material known in the art may be used. For example, stainless steel tubing may be Alloy type 316L SS, Special Chemistry per ASTM F138-92 or ASTM F139-92 grade 2. Special Chemistry of type 316L per ASTM F138-92 or ASTM F139-92 Stainless Steel for surgical implants in weight percent is as follows:

1 Carbon (C) 0.03% max. Manganese (Mn) 2.00% max. Phosphorous (P) 0.025% max. Sulphur (S) 0.010% max. Silicon (Si) 0.75% max. Chromium (Cr) 17.00-19.00% Nickel (Ni) 13.00-15.50% Molybdenum (Mo) 2.00-3.00% Nitrogen (N) 0.10% max. Copper (Cu) 0.50% max. Iron (Fe) Balance

Tubular members formed from any number of metals are possible, including cobalt-chromium, titanium, nickel-titanium, tantalum, gold, platinum, nickel-titanium-platinum, and other similar metal alloys, or from a polymeric material known in the art for making stents.

Generally, the barbs, hooks and/or tabs will be formed unitarily with the rest of the supplemental elasticity device, from the same piece of material. This may be done with laser cutting manufacturing techniques known in the art. But it also is contemplated that the barbs, hooks, and/or tabs may be formed independently of the supplemental elasticity device and subsequently attached to it by welding, brazing or another process with the equivalent effect.

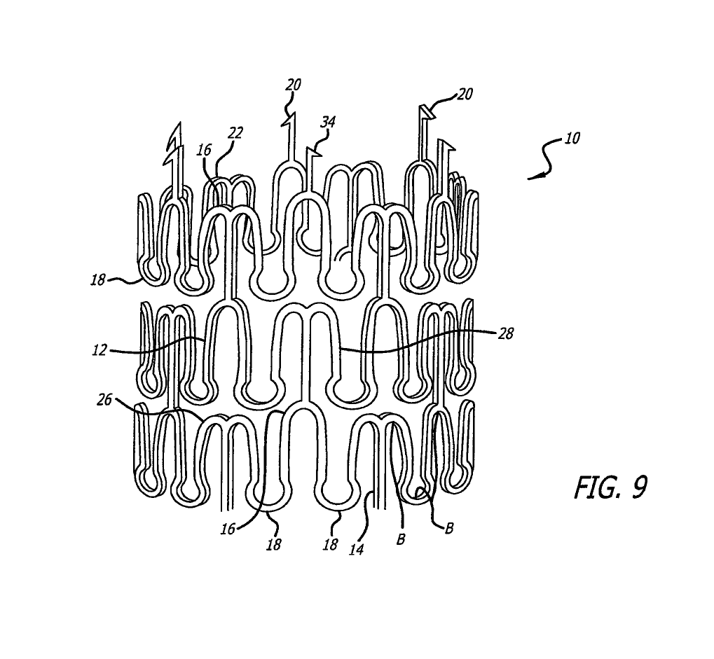

In one embodiment, as shown in FIGS. 8 and 9, it is contemplated that a supplemental elasticity device with the dimensional characteristics disclosed below would be suited for use with a variety of vascular anatomies. It is clear, however, that a supplemental elasticity device with other dimensions might be equally useful. Preferably, length L of the supplemental elasticity device, exclusive of barbs 20, is in the range of about 150 mm-300 mm long, although a wide variety of different widths, lengths and other dimensions may be selected within the scope of the invention.

In one embodiment, it is desirable for connecting members 14 to have a transverse cross-section similar to the transverse dimensions of the undulating components of the expandable bands. As one example, the shaft of each barb may have a length C of approximately 0.05 inch (1.3 mm) and diameter (or width) D of approximately 0.008 inch (0.2 mm). The barbs may have width E of approximately 0.03 inch (0.8 mm). As stated, these dimensions are only examples, and the selection of the most appropriate dimensions in a particular clinical situation may vary considerably from patient to patient.

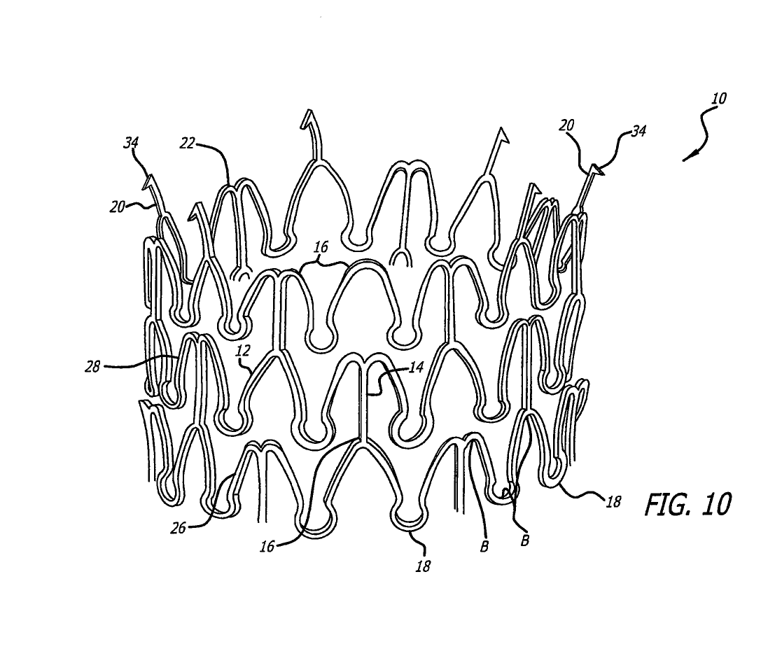

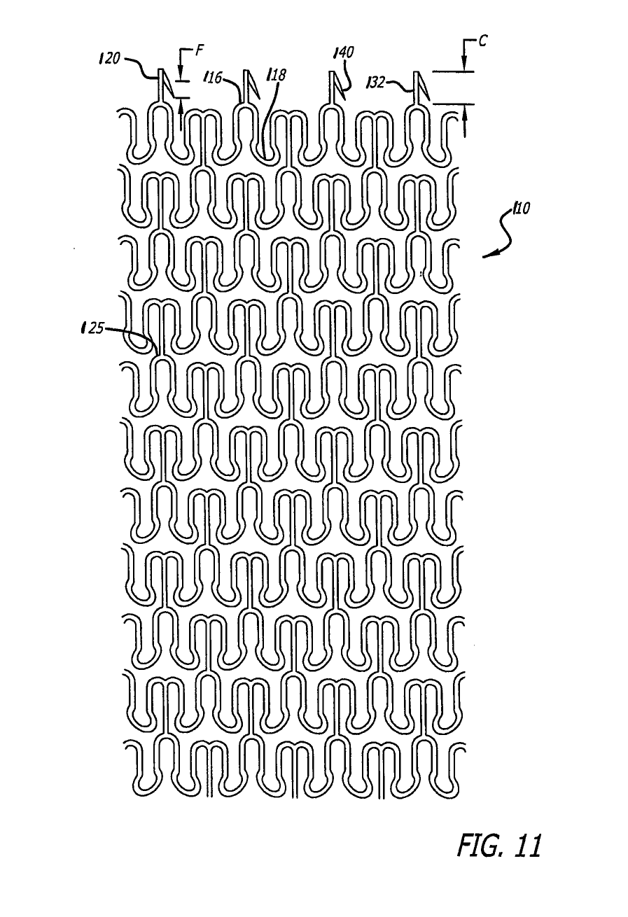

Features of one presently preferred embodiment of the supplemental elasticity device are such as to allow the supplemental elasticity device to be uniformly expanded in a radial direction, as illustrated in FIG. 10, both to a significant degree and without large variation in the level of diametric expansion of each cylindrical ring. The cylindrical rings 12 are transverse to the longitudinal axis of the finished supplemental elasticity device, and the varying degrees of curvature between peaks 16 and valleys 18 tend to equalize the stresses experienced by the supplemental elasticity device during expansion, so that the peaks and valleys of each band deform radially with substantial uniformity upon application of an expansion force. This structure permits the supplemental elasticity device to increase from an initial, small diameter to any number of larger diameters (see FIG. 10). When the interconnections 14 between two cylindrical rings are aligned with the interconnections between all other cylindrical rings, such that the attachment is accomplished by traversing the distance between the peaks 16 of consecutive cylindrical rings, the serpentine pattern of each cylindrical ring is in phase with the pattern of every other ring.

This manner of connection of the cylindrical rings thus minimizes the degree to which the supplemental elasticity device will be shortened or will contract along its longitudinal axis when it is expanded radially about the longitudinal axis. This configuration also limits twisting of the supplemental elasticity device upon expansion and it enhances more uniform expansion. The in-phase cylindrical ring patterns further are thought to reduce the likelihood that the supplemental elasticity device or any portion of it will recoil, or collapse back to its starting diameter after deployment.

A wide variety of different designs may be utilized to form a supplemental elasticity device according to the present invention, including a wide range of stent and/or spring designs. For example, the number and orientation of connecting members of a supplemental elasticity device can be varied in order to maximize the desired longitudinal flexibility of the supplemental elasticity device structure both in the unexpanded and in the expanded condition. Flexibility is advantageous during deployment of the supplemental elasticity device because it improves the ease and safety with which the combination can be delivered through the vascular system to the aneurysm.

Following affixation of the supplemental elasticity device to the arterial wall, longitudinal flexibility minimizes alteration of the natural physiology of the artery due to the implant and helps to maintain compliance of portions of the vessel. The discrete bands in the examples of FIGS. 8-10 also typically have the capacity to rotate slightly with respect to each other without causing any significant alteration of the basic cylindrical structure of the supplemental elasticity device. Accordingly, the cylindrical rings and connections cumulatively result in a supplemental elasticity device that is flexible along its length or longitudinal axis, but which provides uniform expansion and is stable and resistant of collapse. The reticulated structure supplied by the patterning allows for the perfusion of arterial blood into the region of the arterial wall to which elements 18 are attached to anchor the supplemental elasticity device or devices in place. Such perfusion promotes assimilation of the synthetic prostheses by the artery and, more generally, healing of the treated site.

The more uniform radial expansion of this particular embodiment results in a supplemental elasticity device 10 that can be expanded to a large diameter without substantial out-of-plane twisting, because no high stresses are concentrated in any one particular region of pattern 24. Rather, the forces are evenly distributed among the peaks 16 and valleys 18, allowing the cylindrical rings 12 to expand uniformly. Minimizing the out-of-plane twisting experienced by the supplemental elasticity device during delivery and deployment of the supplemental elasticity device also carries with it the benefit of minimizing the risk of thrombus formation.

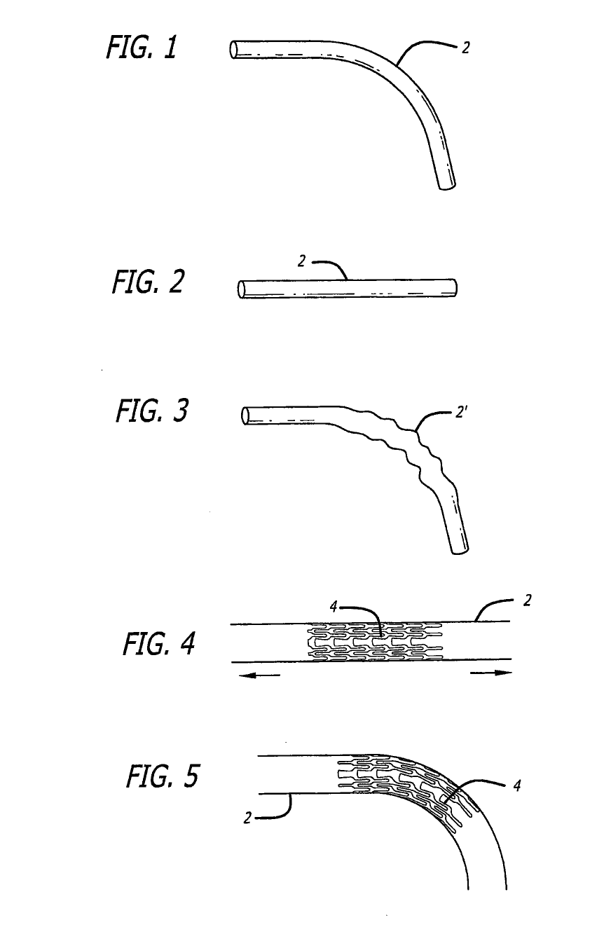

Attachment elements 20 can be provided in a variety of shapes and configurations to ensure adequate attachment of one or more supplemental elasticity devices 10 while the supplemental elasticity device is assimilating into the arterial wall of the artery through endothelial tissue growth. In FIG. 1, attachment elements 120 are in the form of bent spikes, having sharply pointed tips 140 at the end of the shafts 132. When supplemental elasticity device 110 is expanded, some of peaks 116 or valleys 118 of cylindrical ring, or portions thereof, may tip outwardly, becoming embedded in the arterial wall and forcing the spikes also to become lodged in the vessel, thus aiding in retaining the supplemental elasticity device in place as the supplemental elasticity device becomes permanently implanted.

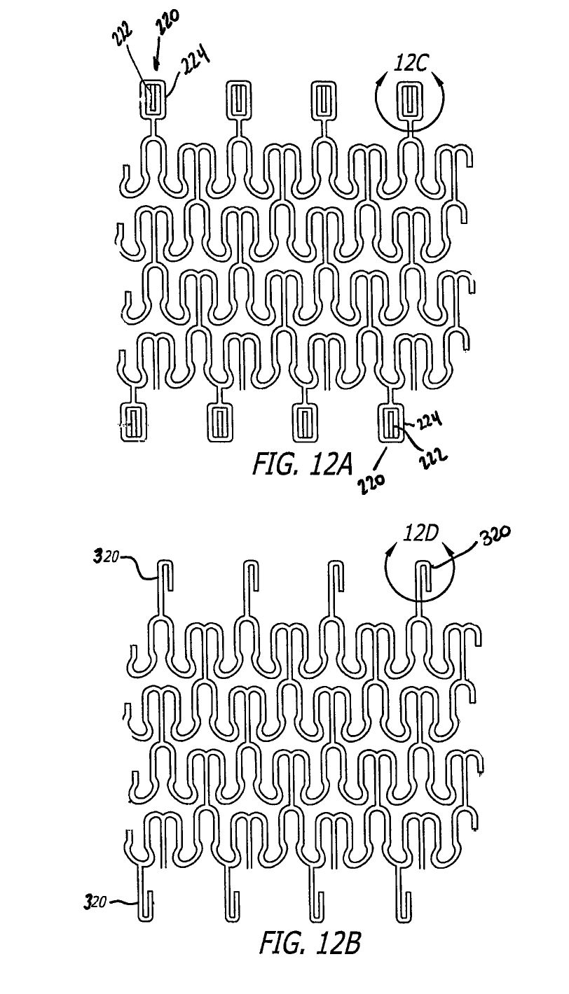

Still other embodiments of the supplemental elasticity device of the invention are shown in FIGS. 12A and 12B. In FIG. 12A, attachment elements 220 are configured as tabs instead of barbs, hooks or spikes. FIG. 12C is a detail view of one such tab member 220, having a tab 234 that protrudes outwardly toward the wall of the lumen. The tab member 220 may be laser cut together with the other portions of the supplemental elasticity device. The tab member 220 would then be bent outwardly in a subsequent step. This particular embodiment has an outer frame 224 forming a circumference of the tab member, and the tab 222 extending inwardly from the outer frame 224.

FIG. 12B illustrates a hook member 320 that serves to attach the supplemental elasticity device to the wall of the lumen. Referring to FIG. 12D, the hook member 320 typically includes a tab portion 322 that extends outwardly from the hook member and toward the wall of the lumen. As with the tab member of FIG. 12C, the hook member 320 may be cut together with the other portions of the supplemental elasticity device. The tab portion 322 of the hook would then be bent in a subsequent step.

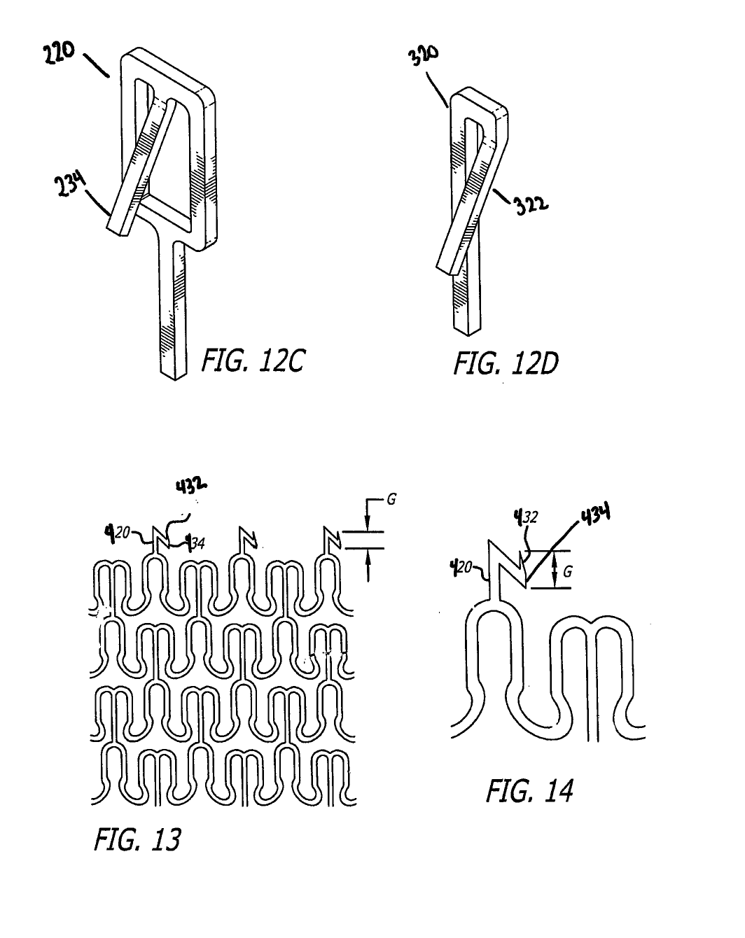

Another embodiment of attachment elements 420 is illustrated in FIGS. 13 and 14. Barbs 434 with dimensions similar to those of the barbs of FIG. 8 are provided, however, the barbs further are equipped with tail portions 432, which have length G of approximately 0.05 inch (1.3 mm), in one embodiment. Again, the dimensions set forth herein only are for illustration purposes and will vary widely depending upon the application and the patient. It can be readily appreciated that many other types of attachment elements may be affixed to or formed unitarily with the supplemental elasticity device body without departing from the scope of the invention. For example, multiple barbs can be provided spaced apart from each other on the shaft of a single attachment element for more than one anchor site per element. The angles formed between the shafts and barbs can vary and can be selected so as to best accomplish the anchoring function in a given application.

Many other shapes and configurations are contemplated that are designed to optimize the attachment of the end of the supplemental elasticity device to the arterial wall while the healing process is taking place to assimilate the supplemental elasticity device into the vessel by endothelial tissue growth. The present invention therefore encompasses a wide range of particular structures, which may vary in configuration, but have in common that they act as a spring-like member that can provide supplemental elasticity to the desired artery.

It is presently preferred to manufacture supplemental elasticity devices according to the present invention in the desired pattern by means of a machine-controlled laser. As general background, a machine-controlled laser cutting system is disclosed in U.S. Pat. No. 5,780,807 to Richard J. Saunders and is incorporated herein by reference. Thin tubing is placed in a rotatable collet fixture of a machine-controlled apparatus for positioning the tubing relative to the laser. According to machine-encoded instructions, the tubing is rotated and moved longitudinally relative to the laser, which is also machine-controlled. The laser selectively removes material from the tubing by ablation and a pattern is cut into the tube. The tube is therefore cut into the discrete pattern of the finished supplemental elasticity device. Hooks, spikes, barbs, and/or tabs at one or more ends of the device may be bent and/or angled as desired after the laser cutting process.

In some embodiments, it may be desirable to incorporate radiopaque markers to identify the position of the supplemental elasticity device assembly during deployment. A supplemental elasticity device 10, for example, can be coated with a metal film that is radiopaque, such as gold, silver, platinum, tantalum and the like. One method of coating the supplemental elasticity device of the present invention with a radiopaque marker is disclosed in U.S. Pat. No. 5,725,572, issued on Mar. 10, 1998 and which is incorporated herein by reference.

A supplemental elasticity device according to the present invention may be delivered to the appropriate position in the popliteal artery via any suitable delivery method known in the art. Various means have been provided to deliver and implant stents, for example. One method frequently described for delivering a stent made of a non-superelastic material to a desired intraluminal location includes mounting the expandable stent on an expandable member, such as a balloon, provided on the distal end of an intravascular catheter, advancing the catheter to the desired location within the patient's body lumen, inflating the balloon on the catheter to expand the stent into a permanent expanded condition and then deflating the balloon and removing the catheter. This general approach may be used to deliver non-superelastic embodiments of a supplemental elasticity device according to the present invention to the area of the patient's knee where supplemental elasticity is desired.

For embodiments of the present invention in which superelastic materials are used, the supplemental elasticity device will typically be self-expanding. Consequently, a balloon is not required with respect to superelastic embodiments of the present device. Delivery systems for delivering self-expanding embodiments of the device are known in the art. The following discussion, then, applies to non-superelastic embodiments of a supplemental elasticity device.

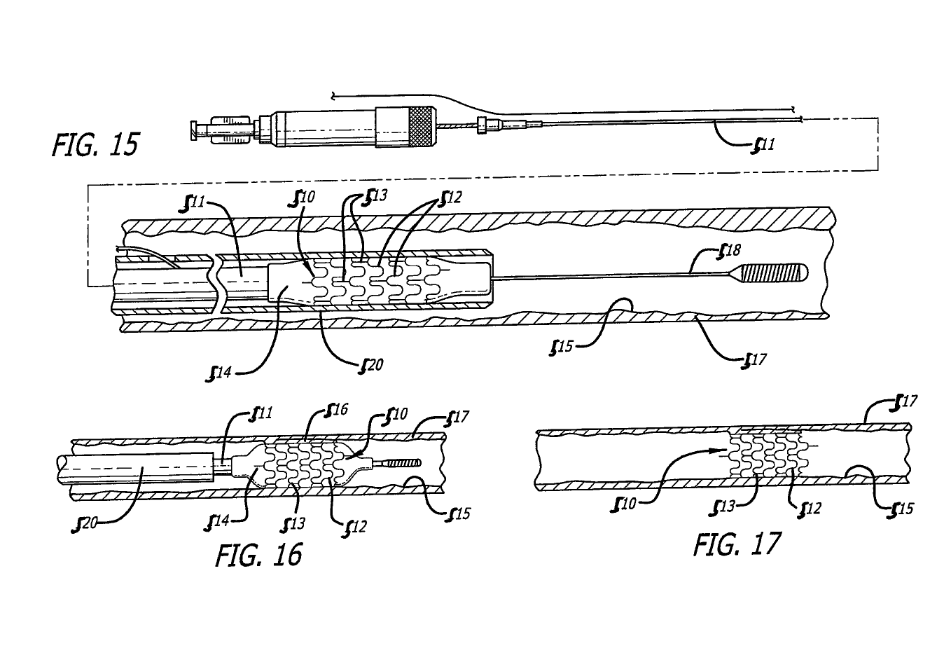

Referring to FIG. 15, the typical delivery catheter 511 onto which a supplemental elasticity member 510 is mounted is essentially the same as a conventional balloon dilatation catheter for angioplasty procedures. The balloon 514 may be formed of suitable materials such as polyethylene, polyethylene terephthalate, polyvinyl chloride, nylon and ionomers such as Surlyn® manufactured by the Polymer Products Division of the Du Pont Company. Other polymers may also be used. In order for the supplemental elasticity member to remain in place on the balloon during delivery to the desired site within the popliteal artery 515, the supplemental elasticity member is compressed onto the balloon. A retractable protective delivery sheath 520 may be provided to further ensure that the supplemental elasticity member stays in place on the expandable portion of the delivery catheter and prevent abrasion of the body lumen by the open surface of the supplemental elasticity member during delivery to the desired arterial location. Other means for securing the supplemental elasticity member onto the balloon may also be used, such as providing collars or ridges on the ends of the working portion, i.e., the cylindrical portion, of the balloon.

The delivery of the supplemental elasticity member 510 is accomplished in the following manner. Referring to FIG. 15, the supplemental elasticity member is first mounted onto the inflatable balloon 514 on the distal extremity of the delivery catheter 511. The balloon is slightly inflated to secure the supplemental elasticity member onto the exterior of the balloon. The catheter-supplemental elasticity member assembly is introduced within the patient's vasculature in a conventional Seldinger technique through a guiding catheter (not shown). A guide wire 518 is disposed across the target arterial section and then the catheter/supplemental elasticity member assembly is advanced over the guide wire within the artery 515 until the supplemental elasticity member is positioned in the target area. The balloon of the catheter is expanded, expanding the supplemental elasticity member against the artery, which is illustrated in FIG. 16. While not shown in the drawing, the artery is preferably expanded slightly by the expansion of the supplemental elasticity member to seat or otherwise fix the supplemental elasticity member to prevent movement.

The supplemental elasticity member 510 holds open the artery 515 after the catheter 511 is withdrawn, as illustrated by FIG. 17. Due to the formation of the supplemental elasticity member from an elongated tubular member, the undulating component of the cylindrical elements of the supplemental elasticity member is relatively flat in transverse cross-section, so that when the supplemental elasticity member is expanded, the cylindrical elements are pressed into the wall of the artery and, as a result, do not interfere with the blood flow through the artery. The cylindrical elements 512 of the supplemental elasticity member, which are pressed into the wall of the artery, will eventually be covered with endothelial cell growth which further minimizes blood flow interference. The undulating portion of the cylindrical elements provides good tacking characteristics to prevent supplemental elasticity member movement within the artery. Furthermore, the closely spaced cylindrical elements at regular intervals provide uniform support for the wall of the artery and, consequently, are well adapted to tack up and hold in place small flaps or dissections in the wall of the artery.

Attachment elements or hooks are provided on the most distal end of the supplemental elasticity device, which hooks ultimately will attach the supplemental elasticity device to regions in the arterial wall. If desired or necessary to achieve a more secure attachment, hooks also can be provided on the proximal end of supplemental elasticity device for attaching to the arterial wall. The hooks anchor the supplemental elasticity devices while the implantation process is on going, and before the body has naturally assimilated the combination through intergrowth of endothelial cells.

When the balloon is inflated by the pressurized fluid or gas source external to the patient, radial forces accompanying expansion of the balloons are applied to expand the supplemental elasticity device (or devices, as several supplemental elasticity devices may be used in sequence) radially outward, pressing both elements against arterial wall. Hooks and/or tabs provided on the supplemental elasticity device become embedded in the arterial wall, to anchor the supplemental elasticity device against downstream arterial pressure while the healing process takes place.

It is noted that various other examples of hook and/or tab designs that may be utilitzed in conjunction with the present invention are generally illustrated, for example, in U.S. Pat. Nos. 7,147,662, 6,517,573, 5,843,164, 5,591,197, 5,681,346, and 5,423,885, all of which are hereby incorporated by reference.

It will be apparent to those skilled in the art that the supplemental elasticity device can be used in various vessels of the body. Because the supplemental elasticity device of the present invention has the novel features of attachment elements and the capacity to expand quickly from relatively small diameters to relatively large diameters, the supplemental elasticity device is particularly well suited for implantation in almost any vessel where such supplemental elasticity devices can be used. This feature, coupled with the fact that the supplemental elasticity device does not contract or recoil to any great degree after it is radially expanded, provides a highly desirable support member for other types of endoprosthesis. Other modifications and improvements may be made without departing from the scope of the invention.

What is claimed is:

1. A method for prosthetic support of flaccid arterial segments comprising the steps of:
implanting a supplemental elasticity device into one of a popliteal leg artery and a superficial femoral leg artery; and
attaching the supplemental elasticity device into place within the artery.

2. A method as defined in claim 1, wherein the step of implanting a supplemental elasticity device comprises implanting a device that has a plurality of adjacent cylindrical elements, each having a circumference extending around a longitudinal supplemental elasticity device axis, and each element being substantially independently expandable in the radial direction, the elements being arranged in alignment along the longitudinal supplemental elasticity device axis, at least one interconnecting member extending between adjacent cylindrical elements and connecting them to one another, and a plurality of protrusions on each end of the supplemental elasticity device for attaching the supplemental elasticity device to the body lumen.

3. A method as defined in claim 2, wherein the protrusions are formed of a unitary structure, and the step of attaching the supplemental elasticity device into place includes radially expanding the supplemental elasticity device to extend the protrusions outwardly and engage the wall of the body lumen.

4. A method as defined in claim 1, wherein the supplemental elasticity device includes hooks, and the step of attaching the supplemental elasticity device into place includes engaging a wall of the body lumen with at least one hook.

5. A method as defined in claim 1, wherein the supplemental elasticity device include tabs, and the step of attaching the supplemental elasticity device into place includes engaging a wall of the body lumen with at least one tab.

6. A method as defined in claim 1, wherein the supplemental elasticity device include barbs, and the step of attaching the supplemental elasticity device into place includes engaging a wall of the body lumen with at least one barb.

7. A method as defined in claim 1, wherein the supplemental elasticity device comprises superelastic nickel titanium.

8. A method as defined in claim 1, wherein the supplemental elasticity device comprises at least one of: stainless steel and a biocompatible polymer.

9. A method for prosthetic support of flaccid arterial segments, in which the prosthesis has a plurality of adjacent cylindrical elements each having a circumference extending around a longitudinal supplemental elasticity device axis and each element being substantially independently expandable in the radial direction, the elements being arranged in alignment along the longitudinal supplemental elasticity device axis; the cylindrical elements formed in a generally serpentine wave pattern transverse to the longitudinal axis and containing a plurality of alternating peaks and valleys; at least one interconnecting member extending between adjacent cylindrical elements and connecting them to one another; a plurality of protrusions on each end of the supplemental elasticity device for attaching the supplemental elasticity device to the body lumen; and the protrusions formed of a unitary structure such that upon radial expansion of the supplemental elasticity device the protrusions bend outwardly and engage the body lumen wall, the method comprising the steps of:
implanting the supplemental elasticity device via an over-the-wire procedure into one of a popliteal leg artery and a superficial femoral leg artery; and
attaching the supplemental elasticity device into place at the ends of the device within in the artery.

10. A method as defined in claim 9, wherein the supplemental elasticity device is capable of enduring elongation cycles in which the supplemental elasticity device is elongated up to 15% of its normal length when a leg is straightened, and is then returned to normal length when the leg is bent.

11. A method as defined in claim 9, wherein the supplemental elasticity device is attached to the artery only at the ends of the supplemental elasticity device.

12. A method as defined in claim 9, wherein supplemental elasticity device has a life of at least 620,000 elongation cycles.

13. A prosthetic support for supporting flaccid arterial segments, comprising:
a plurality of adjacent cylindrical elements each having a circumference extending around a longitudinal supplemental elasticity device axis and each element being substantially independently expandable in the radial direction, the elements being arranged in alignment along the longitudinal supplemental elasticity device axis;
the cylindrical elements formed in a generally serpentine wave pattern transverse to the longitudinal axis and containing a plurality of alternating peaks and valleys; at least one interconnecting member extending between adjacent cylindrical elements and connecting them to one another;
a plurality of protrusions on the supplemental elasticity device for attaching the supplemental elasticity device to the body lumen;
wherein the supplemental elasticity device is capable of enduring elongation cycles in which the supplemental elasticity device is elongated up to 15% of its normal length when a patient straightens a leg, and the supplemental elasticity device is then returned to a normal length when the patient bends the knee, throughout a life of at least 620,000 elongation cycles.

14. A prosthetic support as defined in claim 13, wherein the supplemental elasticity device and protrusions are formed of a unitary structure, such that upon radial expansion of the supplemental elasticity device the protrusions bend outwardly.

15. A prosthetic support as defined in claim 13, wherein the protrusions comprise hooks.

16. A prosthetic support as defined in claim 13, wherein the protrusions comprise tabs.

17. A prosthetic support as defined in claim 13, wherein the protrusions comprise spikes.

18. A prosthetic support as defined in claim 13, wherein the protrusions comprise barbs.

19. A prosthetic support as defined in claim 13, wherein the supplemental elasticity device comprises a stent.

20. A prosthetic support as defined in claim 13, wherein the supplemental elasticity device is a spring.

21. A prosthetic support as defined in claim 13, wherein the supplemental elasticity device is formed of at least one of the group consisting of nickel-titanium, stainless steel, and a polymer.

22. A method of manufacturing a prosthetic support of flaccid arterial segments comprising:
cutting a prosthetic support from a tube of material;
cutting protrusions on at least one end of the prosthetic support; and
bending the protrusions to extend outwardly from the prosthetic support.

23. A method as defined in claim 22, wherein the protrusions are bent into hooks.

24. A method as defined in claim 22, wherein the protrusions are bent into barbs.

25. A method as defined in claim 22, wherein the protrusions are bent into angled tabs.

26. A method as defined in claim 22, wherein the step of cutting protrusions comprises cutting members each having a frame that defines an interior, and a tab member extending from the frame toward the interior.

27. A method as defined in claim 26, wherein the step of bending comprises bending the tab member to extend outwardly from the frame.
